(19) United States
(12) Patent Application Publication
(10) Pub. No.: US 2024/0293312 A1
(43) Pub. Date: Sep. 5, 2024

(54) PHARMACEUTICAL COMPOSITIONS OF 3-(4-AMINO-1-OXO-1, 3 DIHYDRO-ISOINDOL-2-YL)-PIPERIDINE-2, 6-DIONE

(71) Applicants: Shah RISHABH, Gujarat Ahmedabad (IN); Bhavsar BHAVESH, Fuquay Varina, NC (US)

(72) Inventors: Shah RISHABH, Gujarat Ahmedabad (IN); Bhavsar BHAVESH, Fuquay Varina, NC (US)

(21) Appl. No.: 18/575,862

(22) PCT Filed: Jun. 30, 2022
(86) PCT No.: PCT/IB2022/056102

§ 371 Date: Jan. 1, 2024

(30) Foreign Application Priority Data
Jun. 30, 2021 (IN) 202121029430

(51) Int. Cl. A61K 9/08; A61K 31/454; A61K 47/10; A61K 47/12

(52) U.S. Cl. CPC A61K 9/08; A61K 31/454; A61K 47/10; A61K 47/12

(57) ABSTRACT

The present invention relates to pharmaceutical compositions comprising 3-(4-amino-1-oxo-1, 3 dihydro-isoindol-2-yl)-piperidine-2, 6-dione dissolved in a liquid vehicle. The present invention also relates to pharmaceutical compositions comprising 3-(4-amino-1-oxo-1, 3 dihydro-isoindol-2-yl)-piperidine-2, 6-dione and one or more pharmaceutically acceptable excipients. The present invention also related to the process for preparing the said liquid composition and its use for treating multiple myeloma and lymphoma.

### FIELD OF INVENTION

The present invention relates to pharmaceutical compositions comprising 3-(4-amino-1-oxo-1, 3 dihydro-isoindol-2-yl)-piperidine-2, 6-dione. The present invention particularly relates to pharmaceutical compositions comprising 3-(4-amino-1-oxo-1, 3 dihydro-isoindol-2-yl)-piperidine-2, 6-dione and one or more pharmaceutically acceptable excipients. The present invention also related to the process for preparing the said composition and its use for treating multiple myeloma and lymphoma.

### BACKGROUND OF INVENTION

The active ingredient 3-(4-amino-1-oxo-1, 3 dihydro-isoindol-2-yl)-piperidine-2, 6-dione which is also known as lenalidomide has a chemical name 3-(4-amino-1-oxo-1, 3-dihydro-2H-isoindol-2-yl) piperidine-2, 6-dione, and is structurally represented by chemical structure as below:

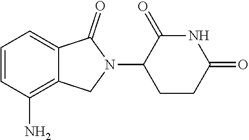 [SMILES: Nc1cccc2c1CN(C1CCC(=O)NC1=O)C2=O]

Lenalidomide, a thalidomide analogue, was initially intended for use as a treatment for multiple myeloma, for which thalidomide is an accepted therapeutic modality, but has also shown efficacy in the hematological disorders known as the myelodysplastic syndromes. The exact mechanism of the immuno-modulatory drugs (e.g., thalidomide, CC-4047/actimid and lenalidomide) is not known. Apart from interfering with the immune system, they are also found to be active for angiogenesis. With myelodysplastic syndromes, the encouraging results of lenalidomide were also obtained in patients with deletion 5q cytogenetic abnormality.

3-(4-amino-1-oxo-1, 3 dihydro-isoindol-2-yl)-piperidine-2, 6-dione or Lenalidomide is a thalidomide analogue. It is an immunomodulatory agent with anti angiogenic and antineoplastic properties. Lenalidomide is off-white to pale-yellow solid powder. It is soluble in organic solvent/water mixtures, and buffered aqueous solvents. Lenalidomide is more soluble in organic solvents and low pH solutions.

Lenalidomide was approved by the U.S. Food and Drug Administration on Dec. 27, 2005 for treating patients with low or intermediate-1 risk MDS with 5q—with or without additional cytogenetic abnormalities.

The drug is commercially marketed in products sold by Celgene Corporation under the brand name REVLIMID™ in the form of capsules having the strengths 2.5 mg, 5 mg, 7.5 mg, 10 mg, 15 mg, 20 mg, and 25 mg.

Lenalidomide has an asymmetric carbon atom and can exist as the optically active forms S(−) and R(+), and is produced as a racemic mixture with a net optical rotation of zero.

International Application Publication No. WO 2005/023192, U.S. Pat. No. 7,465,800 B2, and U.S. Patent Application Publication Nos. 2009/0062343 A1, 2009/0149499 A1, 2009/0149500 A1, 2009/0176832 A1, and 2009/0187023 A1 disclose crystalline lenalidomide hemihydrate, unsolvated crystalline lenalidomide, a solid form of lenalidomide comprising the unsolvated crystalline and amorphous lenalidomide, a crystalline lenalidomide dihydrate, a solid form of lenalidomide comprising dihydrate and amorphous lenalidomide, a solid form of lenalidomide comprising dihydrate and crystalline lenalidomide hemihydrate, a solid form of lenalidomide comprising crystalline lenalidomide hemihydrate and amorphous lenalidomide, a solid form of lenalidomide comprising crystalline Form F and amorphous lenalidomide and polymorphic forms designated as forms A, B, C, D, E, F, G, and H, pharmaceutical compositions comprising the various crystalline forms of lenalidomide, and mixtures of crystalline forms having greater than about 50% crystallinity. Further, processes for preparing the above said forms are also disclosed. International Application Publication No. WO 2009/111948 A1 discloses lenalidomide strong acid salts such as salts with hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embonic acid, enanthic acid, and the like, polymorphic forms of lenalidomide strong acid salts, and processes for the preparation thereof.

Lenalidomide is widely available in form of hard gelatin capsules containing solid powder mixture of lenalidomide with excipients in solid form. Available literature suggests preparation of extemporaneous preparation of liquid suspension form of lenalidomide. However, preparing extemporaneous preparation by emptying the marketed capsule and dissolving the same in water or any other solvent will not result in a proper formulation which can deliver the exact dose requirement and also if the drug is not properly dissolved there is chances of variable drug content in every dose. Second main disadvantage of extemporaneous preparation is stability.

Hence still there is need in the society to have composition of lenalidomide in liquid form with totally dissolved form with physical and chemical stability.

Inventors of present invention have arrived to a composition which is liquid form comprising dissolved 3-(4-amino-1-oxo-1, 3 dihydro-isoindol-2-yl)-piperidine-2, 6-dione.

### OBJECTIVE OF THE INVENTION

The main objective of the present invention is to provide a pharmaceutical compositions of 3-(4-amino-1-oxo-1, 3 dihydro-isoindol-2-yl)-piperidine-2, 6-dione.

The other main objective of the present invention to provide a pharmaceutical compositions of 3-(4-amino-1-oxo-1, 3 dihydro-isoindol-2-yl)-piperidine-2, 6-dione which is safe and effective.

Another objective of the present invention is to provide to provide a pharmaceutical compositions of 3-(4-amino-1-oxo-1, 3 dihydro-isoindol-2-yl)-piperidine-2, 6-dione which is stable.

Another objective of the present invention is to provide to provide a pharmaceutical compositions of 3-(4-amino-1-oxo-1, 3 dihydro-isoindol-2-yl)-piperidine-2, 6-dione which is in a liquid dosage form and having better patient compliance.

Yet another objective of present invention is to provide use of pharmaceutical composition of 3-(4-amino-1-oxo-1, 3 dihydro-isoindol-2-yl)-piperidine-2, 6-dione for treating multiple myeloma and lymphoma.

### SUMMARY OF INVENTION

The main aspect of the present invention is to provide a pharmaceutical compositions of 3-(4-amino-1-oxo-1, 3 dihydro-isoindol-2-yl)-piperidine-2, 6-dione.

Another main aspect of the present invention is to provide a pharmaceutical compositions comprising 3-(4-amino-1-oxo-1, 3 dihydro-isoindol-2-yl)-piperidine-2, 6-dione and one or more pharmaceutically acceptable excipients, wherein the composition is in liquid form and having impurity less than 0.5% after storing for 3 months at 2-8° C.

Another aspect of the present invention is to provide a process for preparing pharmaceutical composition comprising 3-(4-amino-1-oxo-1, 3 dihydro-isoindol-2-yl)-piperidine-2, 6-dione and one or more pharmaceutically acceptable excipients.

### DETAILS DESCRIPTION OF THE INVENTION

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described.

The present invention is all about pharmaceutical compositions comprising 3-(4-amino-1-oxo-1, 3 dihydro-isoindol-2-yl)-piperidine-2, 6-dione dissolved in a liquid vehicle with one or more inactive excipients.

As used herein, the term “pharmaceutically acceptable salt” refers to a salt of a compound that is derived from a variety of physiologically acceptable organic and inorganic counter ions. Such counter ions are well known in the art and include, by way of example only, sodium, potassium, calcium, magnesium, aluminum, lithium and ammonium, for example tetraalkylammonium, and the like when the molecule contains an acidic functionality; and when the molecule contains a basic functionality, salts of organic or inorganic acids, such as hydrochloride, sulfate, phosphate, diphosphate, nitrate hydrobromide, tartrate, mesylate, acetate, malate, maleate, fumarate, tartrate, succinate, citrate, lactate, pamoate, salicylate, stearate, methanesulfonate, p-toluenesulfonate, and oxalate, and the like. In a preferred embodiment the lenalidomide is in the form of free base.

In whole specification chemical name “3-(4-amino-1-oxo-1, 3 dihydro-isoindol-2-yl)-piperidine-2, 6-dione” and lenalidomide conveys the same meaning and can be used interchangeably.

The main embodiment of the present invention is to provide a pharmaceutical composition comprising pharmaceutical compositions comprising 3-(4-amino-1-oxo-1, 3 dihydro-isoindol-2-yl)-piperidine-2, 6-dione and one or more pharmaceutically acceptable excipients, wherein the composition is in liquid form, and having impurity less than 0.5% after storing for 3 month at 2-8° C.

As per one embodiment, the present invention comprising one or more pharmaceutically acceptable excipients which are selected from anti-oxidant, pH modifier and one or more vehicles etc.

### Lenalidomide

Lenalidomide is a thalidomide derivative used to treat multiple myeloma and anemia in low to intermediate risk myelodysplastic syndrome. Lenalidomide is used to treat various types of cancers. It works by slowing or stopping the growth of cancer cells. It is also used to treat anemia in patients with certain blood/bone marrow disorders (myelodysplastic syndromes-MDS). Lenalidomide may lessen the need for blood transfusions.

As per one embodiment, lenalidomide used in the present invention is in the range from 1 to 15% w/w, preferably 1 to 10% w/w, more preferably 1 to 8% w/w, most preferably 1 to 5% w/w.

As per one embodiment, liquid vehicle as used herein is used to dissolve the lenalidomide in such a way that the final liquid composition contains dissolved lenalidomide and also do not get precipitated during shelf life. The choice and concentration of liquid vehicle plays a critical role. The vehicle plays a critical role in getting desired solubility as well as physical and chemical stability.

As per one embodiment, vehicles used in the present invention are selected from Poly Ethylene Glycol 400, Propylene Glycol, water, ethanol and glycerin. In a most preferred embodiment, Poly Ethylene Glycol 400 and Propylene Glycol are used as a vehicles in the present invention.

As per one embodiment each vehicle used in the present invention is in the range from 0.1 to 100% w/w, more preferably 1 to 100% w/w, most preferably 1 to 95% w/w.

As per one embodiment, an “anti-oxidant” is defined as a substance which are used as efficient excipients that delay or inhibit the oxidation process of molecules.

As per one embodiment, an anti-oxidant used in the present invention is selected from butylated hydroxytoluene, butylated hydroxyanisole, sodium metabi-sulfite, lutein, and zeaxanthin. In a most preferred embodiment, butylated hydroxyanisole is used as an anti-oxidant in the present invention.

As per one embodiment, an anti-oxidant used in the present invention is in the range from 0.01 to 3% w/w, preferably 0.1 to 3% w/w, more preferably 0.1 to 2% w/w, most preferably 0.1 to 1% w/w.

As per one embodiment, “pH modifier” can help to maintain the stability of pharmaceutical and can also be used as preservatives. pH modifiers are an important component of aqueous dosage forms.

As per one embodiment, pH modifier used in the present invention is in the range from 0.01 to 3% w/w, preferably 0.1 to 3% w/w, more preferably 0.1 to 2% w/w, most preferably 0.1 to 1% w/w.

As per one embodiment, provided herein is an oral liquid dosage form comprising lenalidomide or pharmaceutically acceptable salt thereof dissolved in a liquid.

As per one embodiment, the liquid composition of the present invention having impurity less than 1%, 0.5%, 0.2% and 0.1% after storing for 3 months at 2-8° C.

As per one embodiment, the process of preparation of liquid pharmaceutical composition of 3-(4-amino-1-oxo-1, 3 dihydro-isoindol-2-yl)-piperidine-2, 6-dione comprises the steps of,
a) weighing Poly Ethylene Glycol 400, Propylene Glycol and glacial acetic acid and mixing them with stirring at room temperature;
b) adding 3-(4-amino-1-oxo-1, 3 dihydro-isoindol-2-yl)-piperidine-2, 6-dione in step (a) solution at room temperature;
c) homogenizing the step (b) suspension until it becomes clear liquid;
d) adding Butylated Hydroxyanisole in step (c) solution with stirring to get final liquid formulation;
e) filling the liquid formulation in a glass vials.

As per one embodiment, homogenization carried out for at 4600 rpm for 45 min and at 1800 rpm for 30 min during process of preparation of lenalidomide liquid formulation.

As per one embodiment, during homogenization process maximum observed temperature was 62.8° C. (at 4600 RPM for 45 minutes) and 50° C. (at 1800 RPM for 30 minutes) due to generation of heat during homogenization process, no external or any other source of heat applied during process of preparation of lenalidomide liquid formulation.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLES

### Example 1: Pharmaceutical Liquid Composition of Lenalidomide

TABLE 1
Pharmaceutical formulation of Lenalidomide

| | | | | | B5 |
|---|---|---|---|---|---|
| | B1 | B2 | B3 | B4 | (Optimized |
| | (% | (% | (% | (% | formulation) |
| Ingredients | w/w) | w/w) | w/w) | w/w) | (% w/w) |
| Lenalidomide | 2.50 | 1.25 | 1.25 | 1.25 | 1.25 |
| Poly Ethylene Glycol 400 | 48.35 | 48.92 | 49.12 | 49.12 | 48.92 |
| Propylene Glycol | 48.35 | 48.92 | 49.12 | 49.12 | 48.92 |
| Butylated Hydroxyanisole | 0.8 | 0.4 | — | — | 0.4 |
| Glacial Acetic Acid | — | 0.5 | 0.5 | 0.5 | 0.5 |

### Procedure:

a) Poly Ethylene Glycol 400, Propylene Glycol and glacial acetic acid were weighed and mixed with stirring at room temperature;
b) 3-(4-amino-1-oxo-1, 3 dihydro-isoindol-2-yl)-piperidine-2, 6-dione was added in step (a) solution at room temperature;
c) the step (b) suspension was homogenized until it becomes clear liquid;
d) Butylated Hydroxyanisole was added in step (c) solution with stirring to get final liquid formulation;
e) the liquid formulation was filled in a glass vials.

### Example 2: Stability Data

The stability of the final composition was performed. The stability was carried out at 2-8° C., 25° C., 30° C. and 40° C. at initially and after 3 months for optimized formulation batch (B5). B5 is optimized batch and shows much better impurity control up to 3M.

The following table shows the result:

| Batch No. | Storage Condition | Assay (%) | Total impurities (%) |
|---|---|---|---|
| B2 | Initial | 101.2 | BRT |
| B3 | Initial | 104.6 | 0.174 |
| B4 | Initial | 102.0 | BRT |
| B4 | 3 M_2-8° C. | 103.1 | 0.543 |
| B5 | Initial | 102.5 | BRT |
| B5 | 3 M_2-8° C. | 103.9 | BRT |
| B5 | 3 M_25° C./60% RH | 104.2 | 0.129 |
| B5 | 3 M_30° C./65% RH | 104.5 | 0.243 |
| B5 | 3 M_40° C./75% RH | 102.4 | 1.563 |

Where,
Reporting Threshold: 0.10%
BRT: Below Reporting Threshold
Total Impurities: sum of all impurities≥BRT (0.10%)

We claim:

1. A pharmaceutical compositions of 3-(4-amino-1-oxo-1, 3 dihydro-isoindol-2-yl)-piperidine-2, 6-dione comprising 3-(4-amino-1-oxo-1, 3 dihydro-isoindol-2-yl)-piperidine-2, 6-dione and one or more pharmaceutically acceptable excipients, wherein the composition is in liquid form and having impurity less than 0.5% after storing for 3 months at 2-8° C.

2. The pharmaceutical compositions of 3-(4-amino-1-oxo-1, 3 dihydro-isoindol-2-yl)-piperidine-2, 6-dione as claimed in claim 1, wherein the one or more pharmaceutically acceptable excipients selected from anti-oxidant, pH modifier and one or more vehicles.

3. The pharmaceutical compositions of 3-(4-amino-1-oxo-1, 3 dihydro-isoindol-2-yl)-piperidine-2, 6-dione as claimed in claim 1, wherein the anti-oxidant is selected from butylated hydroxytoluene, butylated hydroxyanisole, sodium metabi-sulfite, lutein, and zeaxanthin.

4. The pharmaceutical compositions of 3-(4-amino-1-oxo-1, 3 dihydro-isoindol-2-yl)-piperidine-2, 6-dione as claimed in claim 1, wherein the pH modifier is selected from glacial acetic acid, sulfuric acid, hydrofluoric acid, citric acid and hydrochloric acid.

5. The pharmaceutical compositions of 3-(4-amino-1-oxo-1, 3 dihydro-isoindol-2-yl)-piperidine-2, 6-dione as claimed in claim 1, wherein the vehicles are selected from Poly Ethylene Glycol 400, Propylene Glycol, water, ethanol and glycerin.

6. The pharmaceutical compositions of 3-(4-amino-1-oxo-1, 3 dihydro-isoindol-2-yl)-piperidine-2, 6-dione as claimed in claim 1, wherein the composition comprising,
a) 1 to 5% w/w 3-(4-amino-1-oxo-1, 3 dihydro-isoindol-2-yl) piperidine-2, 6-dione,
b) 0.1 to 1% w/w Butylated Hydroxyanisole,
c) 0.1 to 1% w/w glacial acetic acid,
d) 1 to 95% w/w Poly Ethylene Glycol 400 and,
e) 1 to 95% w/w Propylene Glycol

7. The pharmaceutical compositions of 3-(4-amino-1-oxo-1, 3 dihydro-isoindol-2-yl)-piperidine-2, 6-dione as claimed in claim 1, wherein the process of preparation comprises the steps of,
a) weighing Poly Ethylene Glycol 400, Propylene Glycol and glacial acetic acid and mixing them with stirring at room temperature;
b) adding 3-(4-amino-1-oxo-1, 3 dihydro-isoindol-2-yl)-piperidine-2, 6-dione in step (a) solution at room temperature;
c) homogenizing the step (b) suspension until it becomes clear liquid;
d) adding Butylated Hydroxyanisole in step (c) solution with stirring to get final liquid formulation;
e) filling the liquid formulation in a glass vials.
